(19) United States
(12) Patent Application Publication
(10) Pub. No.: US 2024/0077478 A1
(43) Pub. Date: Mar. 7, 2024

(54) SYSTEM FOR RAPID ANALYSIS OF A CAPILLARY BLOOD SAMPLE FROM A SUBJECT, INTENDED FOR DETECTING THE PRESENCE OF AT LEAST ONE ANALYTE IN SAID CAPILLARY BLOOD SAMPLE

(71) Applicant: NG BIOTECH, Guipry-Messac (FR)

(72) Inventors: Milovan STANKOV, Plesse (FR); Milovan STANKOV-PUGES, Rennes (FR)

(21) Appl. No.: 18/262,296

(22) PCT Filed: Jan. 20, 2022
(86) PCT No.: PCT/EP2022/051259

§ 371 Date: Jul. 20, 2023

(30) Foreign Application Priority Data
Jan. 21, 2021 (FR) 2100559

(51) Int. Cl. G01N 33/543; A61B 5/15; G01N 33/558

(52) U.S. Cl. CPC G01N 33/54388; A61B 5/150022; A61B 5/150267; A61B 5/150358; G01N 33/558; A61B 2560/0406; A61B 2560/0443

(57) ABSTRACT

Disclosed is a system for rapid analysis of a capillary blood sample from a subject, intended for detecting the presence of at least one analyte in the capillary blood sample. The rapid analysis system includes an analysis module in which is incorporated at least one immunochromatographic strip. The analysis module includes: a pricking member, suitable for generating a drop of capillary blood; and a collecting member, including a fixed duct, suitable for capillary blood to flow therethrough. The duct has an inlet suitable for collecting the drop of capillary blood, and an outlet, arranged opposite the deposition area and suitable for depositing the drop of capillary blood onto the deposition area, and a container, separate from/independent of the analysis module, in which is packed a buffer solution suitable for implementing the immunochromatographic technique.

### TECHNICAL FIELD OF THE INVENTION

The present invention relates to the technique field of systems for rapid analysis of a capillary blood sample from a subject, intended for detecting the presence of at least one analyte in said capillary blood sample.

### STATE OF THE ART

A rapid diagnostic test, also known as a “rapid screening test”, is a test that rapidly establishes (within a few minutes) the presence of at least one analyte of interest in a biological sample.

This approach conventionally uses the phenomena of chemical reactions using immunochromatography on strips (also known as the “lateral flow test”), which produce a specific colouring for immediate interpretation of the result.

This technique has for interest to be simple, rapid and low cost. Moreover, such tests can be used in the doctors office, but also at the patient's bedside or in the field.

In practice, an immunochromatography strip is conventionally implanted in a box (also called “cassette”) that is adapted to receive biological samples in liquid form.

To detect the presence of at least one analyte in a capillary blood sample, the rapid analysis system has to also include the following elements:
a pricking member, suitable for generating a drop of capillary blood,
a collecting member, suitable for collecting said drop of capillary blood then depositing this drop of capillary blood on an deposition area of the strip, and
a buffer solution suitable for implementing the immunochromatographic technique.

Generally, these different elements of the analysis system are independent, requiring may manipulations.

There however exist analysis modules that integrate such elements as an “all-in-one” device.

Such devices are in practice rather interesting and of simple use. However, they have in particular the drawback of taking up a lot of space for manipulations near the patient.

It would thus be interesting to propose a new rapid analysis system that combines the above-mentioned elements, while preserving simplicity of use and optimized footprint.

### DISCLOSURE OF THE INVENTION

In order to remedy the above-mentioned drawback of the state of the art, the present invention proposes a new system for rapid analysis of a capillary blood sample from a subject, intended for detecting the presence of at least one analyte in said capillary blood sample.

More particularly, the invention proposes a rapid analysis system comprising:
(i) an analysis module into which is incorporated at least one immunochromatographic strip, designed to detect the presence of said at least one analyte by an immunochromatographic technique,
wherein said at least one immunochromatographic strip includes a deposition area intended to receive said capillary blood sample and a capture area intended to detect the presence of said at least one analyte, wherein said analysis module includes:
   a pricking member, suitable for generating a drop of capillary blood, and
   a collecting member, comprising a fixed duct, suitable for capillary blood to flow therethrough,
said duct having an inlet suitable for collecting said drop of capillary blood, and an outlet, arranged opposite said deposition area and suitable for depositing said drop of capillary blood onto said deposition area, and
(ii) a container, separate from/independent of said analysis module, in which is packed a buffer solution suitable for implementing said immunochromatographic technique.

Other non-limiting and advantageous features of the product according to the invention, taken individually or according to all the technically possible combinations, are the following:
the analysis module has an elongated shape, delimited by two ends; the pricking member is implanted at a first end of said analysis module, and the collecting member is implanted at a second end of said analysis module;
the collecting member also includes a through-hole suitable for receiving the buffer solution, arranged opposite the deposition area, advantageously, as the case may be, in said at least one connecting part and opposite the deposition window; the through-hole advantageously includes an annular surface delimited by an inlet edge and an outlet edge, connected by a flared annular surface; preferably, the duct outlet leads to the through-hole; the duct advantageously has a gutter shape, whose downstream outlet is cantilevered/protruding with respect to the through-hole;
the analysis module includes a gripping portion, arranged near the pricking member, advantageously on the connecting part.

According to a preferred embodiment, the analysis module includes a cassette into which is incorporated said at least one immunochromatographic strip; the pricking member and the collecting member are assembled to said cassette through assembly means, for example through fitting means.

Other non-limiting and advantageous features of this preferred embodiment according to the invention, taken individually or according to all the technically possible combinations, are the following:
the pricking member and the collecting member are carried by at least one connecting part, advantageously forming an adapter, which cooperate with the cassette through said assembly means;
the pricking member and the collecting member are carried by a single connecting part, which includes means for assembly to the cassette, or two distinct connecting parts, each including means for assembly to the cassette;
the pricking member cooperates with said at least one connecting part through assembly means;
the cassette has two front walls that are connected by a lateral wall, wherein a first front wall includes two windows, a deposition window, arranged opposite the deposition area of said at least one immunochromatographic strip, and a reading window, arranged opposite the capture area of said at least one immunochromatographic strip, and said at least one connection part has at least a front wall placed on said first front wall of the cassette and a skirt placed on said lateral wall of the cassette; preferably, the pricking member is implanted opposite and in the continuation of the lateral wall of the cassette.

The present invention also relates to the analysis module for a system for rapid analysis of a capillary blood sample from a subject, intended for detecting the presence of at least one analyte in said capillary blood sample.

The analysis module incorporates at least one immunochromatographic strip, designed to detect the presence of said at least one analyte by an immunochromatographic technique.

Said at least one immunochromatographic strip includes a deposition area intended to receive said biological sample and a capture area intended to detect the presence of said at least one analyte.

Said analysis module includes:
a pricking member, suitable for generating a drop of capillary blood, and
a collecting member, comprising a fixed duct, suitable for capillary blood to flow therethrough, said duct having an inlet for collecting said drop of capillary blood, and an outlet for depositing said drop of capillary blood onto said deposition area.

The analysis module is devoid of container in which is packed a buffer solution suitable for implementing said immunochromatographic technique.

Of course, the different features, alternatives and embodiments of the invention can be associated with each other according to various combinations, insofar as they are not mutually incompatible or exclusive.

### DETAILED DESCRIPTION OF THE INVENTION

Moreover, various other features of the invention emerge from the appended description made with reference to the drawings that illustrate non-limiting embodiments of the invention, and wherein:

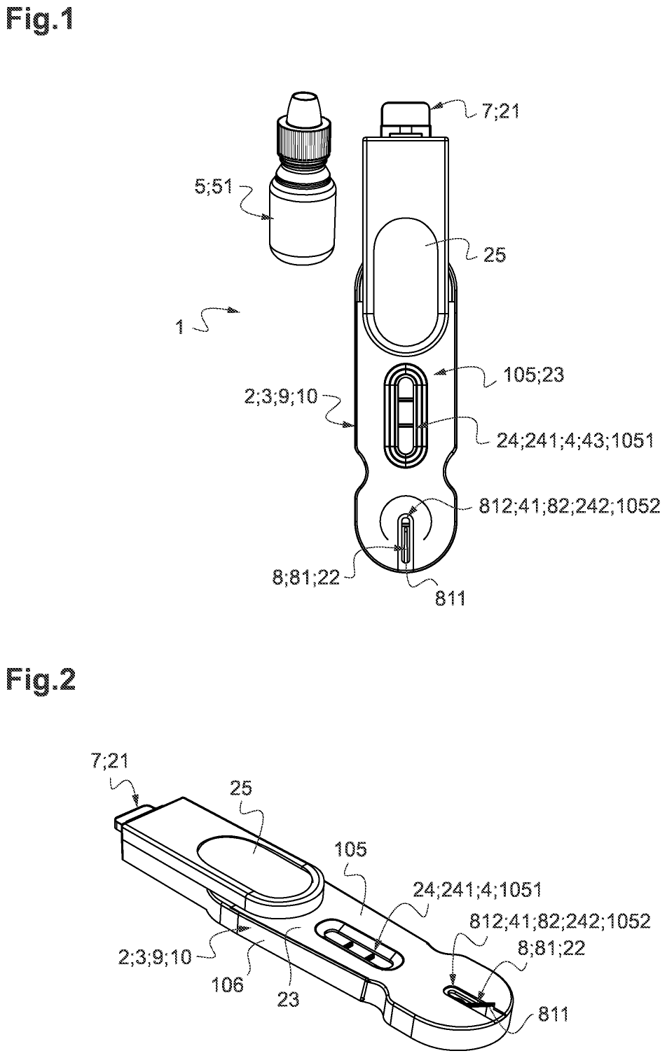

FIG. 1 is a general view of a rapid analysis system according to the invention, wherein the analysis module includes a pricking member and a collecting member, which are carried by a single connecting part assembled to a cassette;

FIG. 2 is a general perspective view of the analysis module according to FIG. 1;

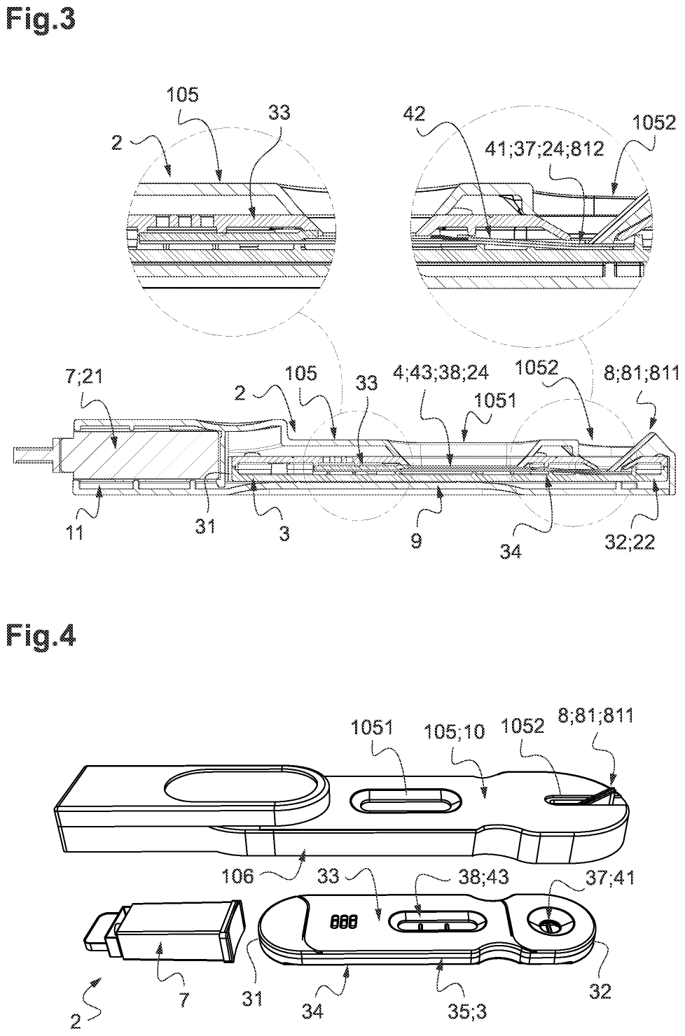

FIG. 3 is a schematic view, along a longitudinal sectional plane, of the analysis module according to FIG. 1;

FIG. 4 is a general, exploded, perspective view of the analysis module according to FIG. 1;

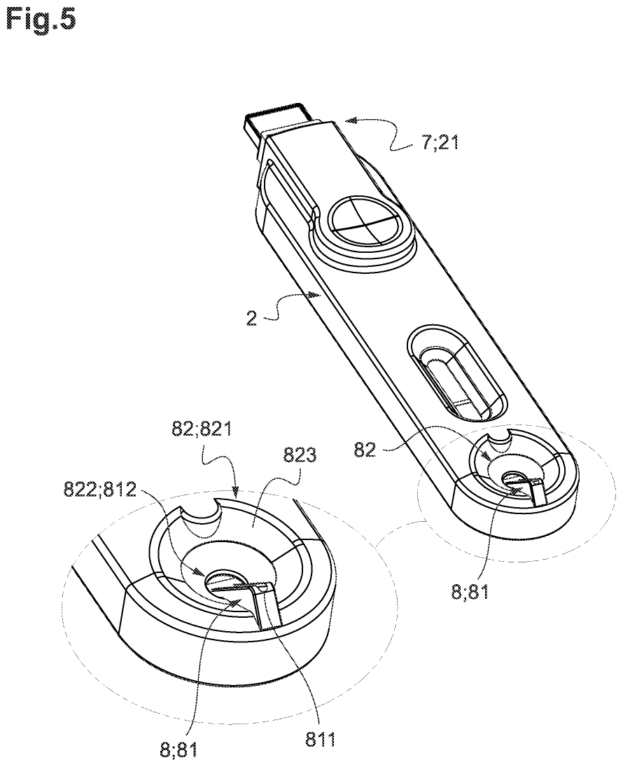

FIG. 5 is a general perspective view of the analysis module according to FIG. 1, including a flared through-hole at the collecting member;

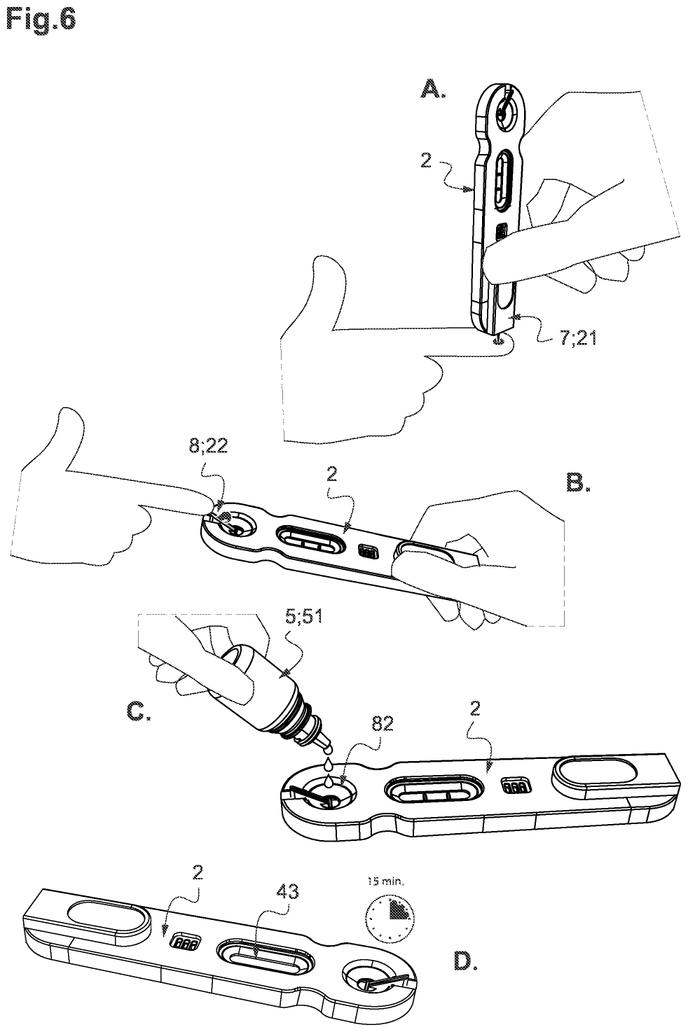

FIG. 6 is a general view illustrating the implementation of the analysis system according to FIG. 1;

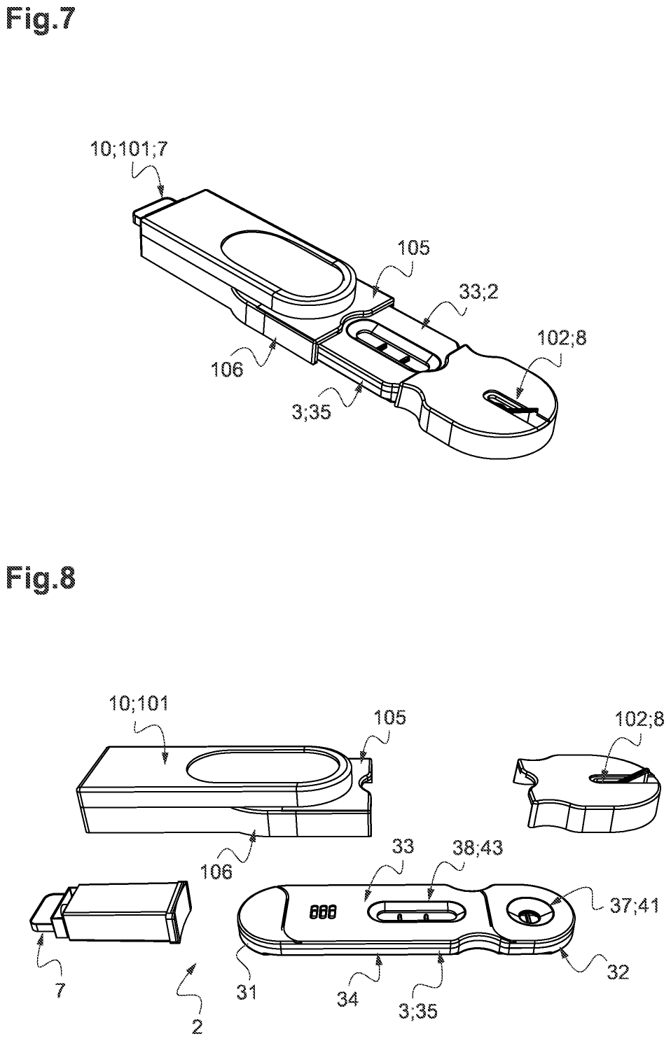

FIG. 7 is a general view of another rapid analysis module according to the invention, wherein the pricking member and the collecting member are carried by two distinct connecting parts assembled to a cassette;

FIG. 8 is a general, exploded, perspective view of the analysis module according to FIG. 7.

It is to be noted that, in these figures, the structural and/or functional elements common to the different alternatives may have the same references.

The rapid analysis system 1 according to the invention, described in relation with the figures, is suitable for rapid analysis of a capillary blood sample E (also called “drop of capillary blood”) from a subject, intended for detecting the presence of at least one analyte A in said capillary blood sample E.

By “detecting”, it is meant qualitative determination (advantageously the presence or the absence), or even quantitative determination, of one or several analytes A in the capillary blood sample E.

By “analyte”, it is meant any chemical, biochemical or biological entity, that is desired to be detected in a capillary blood sample E.

This chemical entity advantageously consists of an entity from the living world, preferably present in humans.

Among the analytes detected by the system and method according to the present invention, mention may be made to proteins, peptides, antibodies, hormones, steroids, antigens derived from infectious agents or tumour cells, infectious agents such as bacteria, viruses or parasites, nucleic acids (DNA or RNA), therapeutic compounds, drugs or antibiotics.

Said at least one analyte A is still preferably selected among the antigens specific of an infectious agent.

By “infectious agent”, it is preferably meant viruses, in particular viruses responsible for pneumopathies, advantageously Coronaviridae, still preferably Orthocoronavirinae or coronaviruses.

The term “coronavirus” encompasses SARS-CoV, MERS-CoV or SARS-CoV-2.

By “capillary blood sample”, it is meant a mixture of blood from arterioles, venules, capillaries and interstitial and intracellular fluid, obtained by capillary puncture.

Such a sample is advantageously obtained by pricking the skin, generally on the finger or the heel.

For that purpose, the rapid analysis system 1 according to the invention comprises:
(i) an analysis module 2 into which is incorporated at least one immunochromatographic strip 4 (visible in FIG. 3) for analysing the capillary blood sample E, and
(ii) a container 5, separate from/independent of said analysis module 2, in which is packed a buffer solution 51 suitable for implementing the immunochromatographic technique.

According to the invention, the analysis module 2 also includes:
a pricking member 7, suitable for generating a drop of capillary blood, and
a collecting member 8, suitable for collecting said drop of capillary blood and suitable for depositing said drop of capillary blood on the immunochromatographic strip 4.

In other words, the analysis module 2 forms a device including at least one support part, or even an assembly of at least two support parts, advantageously made of a plastic material, which carries said at least one immunochromatographic strip 4, the pricking member 7 and the collecting member 8.

Generally, and according to a preferred embodiment, the analysis module 2 has an elongated shape, advantageously a generally parallelepiped shape.

This analysis module 2 is delimited by two ends 21, 22, which are longitudinally opposite to each other.

This analysis module 2 also advantageously has an upper side 23 that includes at least one through-hole 24 in communication with said at least one immunochromatographic strip 4.

The pricking member 7 and the collecting member 8 are advantageously distributed at the two ends 21, 22 of the analysis module 2:
the pricking member 7 is implanted at a first end 21 of the analysis module 2, and
the collecting member 8 is implanted at a second end 22 of the analysis module 2.

It is then just necessary to turn the analysis module 2 upside down to generate the drop of capillary blood then to collect the latter.

Generally, according to the invention, the analysis module 2 is devoid of container 5 in which is packed a buffer solution 51 suitable for implementing the immunochromatographic technique.

This technical feature contributes to an optimum footprint of the analysis module 2.

The analysis module 2 also advantageously includes a gripping portion 25, arranged near the pricking member 7 and at the upper side 23, advantageously carried by a connecting part 10 that will be described hereinafter.

This gripping portion 25 is useful for handling the analysis module 2, in particular when the pricking member 7 is used.

Immunochromatographic Strip

The immunochromatographic strip 4, also called “capillary diffusion means”, is designed to detect the presence of said at least one analyte A by an immunochromatographic technique.

These immunochromatographic strips 4 are formed of any means constituting or acting as a unit of continuous capillary diffusion, by lateral migration (i.e. perpendicular to the thickness of the capillary material(s) implemented for the capillary diffusion).

This capillary diffusion means advantageously consists of a porous solid support enabling the migration of a liquid by simple capillary diffusion.

The porosity of this support enables the capillary diffusion (or lateral migration) of the sample and/or the reagents at the liquid or wet state.

Such capillary diffusion means are very widely used, in particular in all the lateral-migration immunochromatographic techniques.

Such an immunochromatographic strip 4 here consists of a support elongated along the direction and/or orientation of the capillary diffusion (lateral migration).

The immunochromatographic strip 4 can be consisted of:
a single and same capillary or porous material, or
several different capillary or porous elements or materials, suitably arranged with respect to each other (for example, with overlap), to obtain a continuity of capillary flow from an element or a material to another, along the capillary diffusion direction.

Such an immunochromatographic strip 4 determines a direction and orientation of capillary diffusion of any liquid that is received or deposited at an upstream end, and that then moves towards a downstream end of the immunochromatographic strip 4.

By way of example, the immunochromatographic strip 4 can be consisted of various immunochromatographic supports, for example cellulose, nylon, nitrocellulose, polyethylene or glass fibre.

As described in relation with FIG. 3, the immunochromatographic strip 4 includes different successive areas, in the upstream to downstream capillary migration direction, i.e. at least:
a deposition area 41, intended to receive the capillary blood sample E and the buffer solution 51,
a release area 42, which comprises at least one detection reagent conjugated with a visible and/or measurable marker, said detection reagent being capable of moving due to the migration of the buffer solution 51 along the immunochromatographic strip 4, and
at least one capture area 43 that comprises at least one capture reagent, immobilized on the immunochromatographic strip 4, to detect said at least one analyte A.

The deposition area 41 and/or said at least one capture area 43 are advantageously accessible through at least one through-hole 24 arranged in the upper side 23 of the analysis module 2.

In other words, the upper side 23 of the analysis module 2 advantageously includes:
a first through-hole 241, opposite said at least one capture area 43, for reading the analysis, and
a second through-hole 242, opposite said deposition area 41, for depositing the capillary blood sample E and the buffer solution 51.

The release and capture areas advantageously consist of a transverse line or strip (extending perpendicular to the migration direction), having for example a width between 1 and 2 mm and a surface between 3 and 5 mm².

Generally, the “detection reagent” or the “capture reagent” consist of any chemical, biochemical or chemical entity, which is capable of binding specifically to form a complex enabling the determination of said analyte in the capillary blood sample E.

The detection reagent and/or the capture reagent are also so-called “binding” reagents.

Such binding reagents, enabling the determination of at least one analyte in the capillary blood sample E, are well known and can be selected as required for implementing the invention.

These binding reagents are advantageously selected from those which are capable of binding specifically with said analyte and/or of binding specifically with each other.

According to the test format implemented, the complementary binding reagents are intended to form different complexes:
the binding reagents are capable of binding concomitantly with the analyte, to form a sandwich format test, or
one of the binding reagents (detection or capture) is capable of binding to the analyte but also to the other binding reagent (respectively, capture or detection) to form a test in the competition format.

By “binding” or “bond”, it is advantageously meant any weak bond of the antigen/antibody type.

The binding reagents are advantageously selected from antibodies and antigens.

The analyte and the binding reagent thus typically form a couple capable of binding specifically with each other, as for example an antigen/antibody couple.

Therefore, if the analyte is an antigen or hapten, one at least of the binding reagents (the detection reagent and/or the capture reagent) is advantageously an analyte-specific antibody.

By “analyte-specific antibody”, it is meant an antibody capable of binding specifically with the analyte into a bond of the antigen/antibody type.

It is typically a polyclonal antibody or a monoclonal antibody, having a strong affinity with the analyte. Preferably, it is a monoclonal antibody.

If the analyte is an antibody, one at least of the binding reagents is advantageously the antigen recognised by the antibody.

The detection reagent(s) are advantageously conjugated to a visible and/or measurable marker, advantageously a particulate marker.

By “visible and/or measurable marker”, it is meant any marking enabling a direct or indirect detection with the naked eye, or using an apparatus, due to the emission of a signal at said at least one capture area 43.

The signal is for example a fluorescence, a colouration, the presence of an isotope or a magnetic signal.

Examples include coloured particle markers such as colloidal gold, or fluorescent markers, coloured latex particles, fluorescent latex particles and the avidin and streptavidin conjugated particles.

Particulate markers, coloured or fluorescent, thus consist of small particles that are insoluble in water and therefore form suspensions, dispersions or solutions in liquid phase.

Among the markers allowing a direct naked-eye observation, mention can also be made to dextran-type markers (Hansen T. M., IVD Technology 4, 35-40, 2003). The binding reagent is then conjugated to a dextran chain (polysaccharide derivative) carrying fluorophores.

The markers can also consist of enzymes (in particular, alkaline phosphatase or AP, horseradish peroxidase or HRP), dyes or chemiluminescent compounds (in particular, fluorescein isothiocyanate or FITC).

To increase the sensitivity, a labelled antibody can be used, for example, according to techniques known by the person skilled in the art for indirect detection, such as for example a biotinylated antibody, allowing indirect detection by the formation of avidin-biotin and streptavidin-biotin entities.

This labelled and biotinylated antibody can also either be already directly deposited on a test-line, in the capture area, to increase the sensitivity, or be deposited with the specific detection antibody, to increase the time of contact and also the sensitivity, in particular, for example, due to the number of binding sites.

For its part, in the capture area 43, the analyte-specific capture reagent is immobilized on the solid support by techniques known by the person skilled in the art.

This capture reagent is immobilized in such a way that it is not mobile when wet.

This immobilisation may be made for example by absorption or by covalent coupling.

In the system according to the figures, the detection reagents and the capture reagents are selected among the reagents suitable for detect the presence of said at least one analyte selected among the antigens, preferably specific of an infectious agent, preferably viruses, in particular viruses responsible for pneumopathies, advantageously Coronaviridae, still preferably Orthocoronavirinae or coronaviruses.

The detection reagents and the capture reagents are advantageously selected, without being in any way limiting, among:
antibodies, advantageously selected among the anti-IgG antibodies (human) and the anti-IgM antibodies (human), preferably directed against the SRAS-Cov-2,
antibodies specific of a micro-organism, advantageously of a virus, preferably SRAS-Cov-2, and
adapted recombinant proteins.

In a preferred embodiment, the capture area 43 can also comprise a control capture reagent.

This control capture reagent provides a positive control to ensure the effective capillary diffusion of the liquid sample from the deposition area 41 to the capture area 43.

This control capture reagent is permanently immobilized downstream from the “analyte” capture reagents (“Control line”).

It may be for example an antibody binding to the detection reagent(s).

As an alternative, this control capture reagent is independent of the analyte A and is simply used to check the diffusion of the liquid sample along the immunochromatographic strip 4 (for example by capture of a marked control reagent).

Buffer Solution

The buffer solution 51 is suitable for rinsing the collecting member 8 of the analysis module 2 and being mixed with the capillary blood sample E for implementing the immunochromatographic technique.

This buffer solution 51 is in particular intended for migrating along the chromatographic strip 4 in such a way as to cause, or at least facilitate, the migration of the capillary blood sample E (and in particular of said at least one analyte A).

The buffer solution 51 is for example selected among the diluents composed of a buffered saline solution. It can also comprise a detergent or any other component necessary in particular for the migration or the reactions on the immunochromatographic strips 4.

To control the added quantity of this buffer solution 51, the buffer solution 51 is advantageously packaged in a container 5 which includes a dropper.

Pricking Member

The pricking member 7, also called “self-prick” or “lancet”, is conventional per se and single-use.

It advantageously consists of a single-use device serving to prick or puncture the skin, comprising a surgical blade or a needle that retracts irreversibly after use.

Such a pricking member 7 is suitable for generating a capillary blood flow at the pricking point.

Such a pricking member 7 is advantageously suitable for making a transcutaneous pricking, advantageously of 2.2 to 2.5 mm depth, generally on the lateral edges of the fingertip or on the heel.

Collecting Member

The collecting member 8 comprises a fixed duct 81, suitable for capillary blood to flow therethrough.

By “fixed”, it is advantageously understood a duct 81 that is immobile on the collecting member 8, with an identical position for collecting the drop of capillary blood and for depositing this drop of capillary blood on the immunochromatographic strip 4.

The duct 81 includes two ends:
an inlet 811, suitable for collecting the drop of capillary blood, and
an outlet 812, arranged opposite the deposition area 41 of the immunochromatographic strip 4 and suitable for depositing the drop of capillary blood onto this deposition area 41.

The capillary blood sample is thus intended to flow naturally, advantageously by capillarity and/or by gravity along the duct 81, from the inlet 811 to the outlet 812.

The duct 81 is advantageously suitable for collecting a volume of capillary blood suitable for the analysis, for example of the order of 10 μL.

The duct 81 has here advantageously a gutter or channel shape, with advantageously a generally U or V-shaped cross-section, having advantageously an upper longitudinal opening (opening away from the immunochromatographic strip 4).

This embodiment is interesting for rinsing the duct 81 with the buffer solution 51.

The collecting member 8 also advantageously includes a through-hole 82 suitable for receiving the buffer solution 51, arranged opposite the deposition area 41 of the immunochromatographic strip 4 and advantageously leading to the outlet 812 of the duct 81.

This through-hole 82 advantageously corresponds to the above-mentioned second through-hole 242, arranged in the upper side 23 of the analysis module 2.

The through-hole 82 can take different shapes, adapted to the passage of the buffer solution 51.

According to an embodiment illustrated in FIG. 5, the through-hole 82 advantageously has an annular surface 823 delimited by two edges:
a circular inlet edge 821, and
a circular outlet edge 822.

The two edges 821, 822 are connected by a flared annular surface 823, for example in the shape of a truncated cone, the cross-section of which increases from the outlet edge 822 (small diameter) to the inlet edge 821 (great diameter).

Such a through-hole 82, in the general shape of a funnel, is useful for facilitating/concentrating the supply of buffer solution 51 to the deposition area 41.

Preferably, the outlet 812 of the duct 81 leads to the through-hole 82. This approach is interesting for rinsing the duct 81 with the buffer solution 51.

The downstream outlet 812 of the duct 81 is advantageously cantilevered/protruding with respect to the through-hole 82 and in particular the outlet edge 822 thereof (see in particular FIG. 3).

This shape advantageously ensures an optimum deposition of the sample, without contact with the outlet 812 of the duct 81.

The duct 81 advantageously extends further along the flared annular surface 823, between these two edges 821, 822.

This arrangement also contributes to optimizing its rinsing with the buffer solution 51.

The inlet 811 of the duct 81 advantageously protrudes with respect to the through-hole 82 (and protrudes with respect the upper side 23 of the analysis module 2), to facilitate the collection of the capillary blood sample E.

Pricking Member and Collecting Member Assembled to a Cassette

As illustrated in FIG. 4 in particular, the analysis module 2 advantageously includes a cassette 3 into which is incorporated said at least one immunochromatographic 4.

This cassette 3 is advantageously formed by an assembly of at least two support parts, forming a casing, which are made of a plastic material.

Within this framework, the pricking member 7 and the collecting member 8 are advantageously assembled to this cassette 3 through assembly means 9, for example through fitting means (advantageously through elastic fitting means), which are advantageously carried by a connecting part 10 described hereinafter.

The pricking member 7 and the collecting member 8 thus advantageously form accessories that are added on the cassette 3.

This embodiment has for interest to be able to provide additional functions to a cassette 3, which may be conventional per se, in such a way as to enable the analysis of capillary blood samples without requiring the implementation of a combination of distinct devices.

The cassette 3 of the analysis module 2 has advantageously a generally parallelepiped, elongated shape, delimited by two ends 31, 32 (longitudinally opposite to each other).

The cassette 3 advantageously includes two front walls 33, 34 that are connected by a peripheral lateral wall 35.

The first (advantageously upper) front wall 33 advantageously includes two windows:
a deposition window 37, arranged opposite the deposition area 41 of said at least one immunochromatographic strip 4, and
a reading window 38, arranged opposite the capture area 43 of said at least one immunochromatographic strip 4.

For the assembly thereof, the pricking member 7 and the collecting member 8 are advantageously carried by at least one connecting part 10, advantageously forming an adapter adapted to be assembled to the cassette 3.

Said at least one connecting part 10 then cooperates with the cassette 3 through the above-mentioned assembly means 9.

Generally, the pricking member 7 and the collecting member 8 can be carried by:
a single connecting part 10, which includes means 9 for assembly to the cassette 3 (FIGS. 1 to 6), or
two distinct connecting parts 10, each including means 9 for assembly to the cassette 3 (FIGS. 7 and 8).

A single connecting part 10 is advantageously intended to cooperate with the two ends 31, 32 of the cassette 3.

In the presence of two distinct connecting parts 10 (FIGS. 7 and 8), the pricking member 7 and the connecting part 8 are advantageously each carried by one of said connecting parts 10:
a first connecting part 101 carries the pricking member 7, and
a second connecting part 102 carries the connecting part 8.

These two connecting parts 101, 102 are then intended to be placed at one of the two ends 31, 32 of the cassette 3, respectively.

Generally, said at least one connecting part 10 advantageously includes different walls intended to conform the cassette 3, i.e. at least:
a front wall 105 that is placed on the first front wall 33 of the cassette 3, and
a skirt 106 that is placed on the lateral wall 35 of the cassette 3.

In the case of a single connecting part 10, the front part 105 advantageously includes a through-hole 1051 intended to come opposite the reading window 38 of the cassette 3, to form together the first through-hole 241 opposite said at least one capture area 43 for reading the analysis.

Moreover, the through-hole 82 of the collecting member 8, arranged opposite the deposition area 41, is here advantageously arranged through the connecting part 10 (in particular its front wall 105) and opposite the deposition window 37 of the cassette 3.

In other words, the front part 105 advantageously includes a through-hole 1052 intended to come opposite the reading window 37 of the cassette 3, to form together the second through-hole 242 opposite said at least one deposition area 41.

Generally, the assembly means 9 can consist of fitting means, for example elastic fitting means (for example, ribs), arranged between said at least one connecting part 10 and said cassette 3.

As shown in FIG. 3, the assembly means 9 can also consist of at least one additional and lower added part, which cooperate with said at least one connecting part 10 to form together a casing enveloping the cassette 3.

The cassette 3 is then sandwiched between the front wall 105 of the connecting part 10 and the underlying, additional added part 9.

Moreover, the pricking member 7 advantageously consists of an added part, cooperating with said at least one connecting part 10 through assembly means 11 (FIG. 3), for example elastic assembly means.

The assembly means 11 for the pricking member 7 may also be formed by the above-mentioned added part 9 (see FIG. 3).

The pricking member 7 is advantageously implanted opposite and in the continuation of the lateral wall 35 of the cassette 3, on the side of the first end 31 of said cassette 3 (at the opposite of the deposition window 37).

This pricking member 7 advantageously extends in the longitudinal continuation of the cassette 3 and in the thickness of the cassette 3, ensuring a minimum thickness.

Implementation

In practice and as the case may be, the analysis module 2 is assembled before implementation.

In that respect, the pricking member 7 and the collecting member 8 (carried by at least one connecting part 10) are advantageously added on the cassette 3 containing said at least one immunochromatographic strip 4.

The analysis module 2 is then handled in such a way as to generate a drop of capillary blood by means of the pricking member 7 that is arranged at a first end 21 of the analysis module 2 (item A. of FIG. 6).

A capillary blood sample is then collected by means of the collecting member 8 that is arranged at the second end 22 of this same analysis module 2 (item B. of FIG. 6).

The sample then automatically/naturally flows to the immunochromatographic strip 4, via the duct 81.

Thereafter, a suitable quantity of the buffer solution 51 is added through the through-hole 82 of the collecting member 8 that is provided for that purpose, by means of the container 5 that is separate from/independent of the analysis module 2 (item C. of FIG. 6).

Finally, after a suitable time for the migration of the buffer solution 51, for example from 10 to 20 minutes, the reading of said at least one chromatographic strip 4 is made at the capture area 43 of said at least one immunochromatographic strip 4, through the first through-hole 241, to detect the potential presence of said at least one analyte A in said biological sample E (item D. of FIG. 6).

The reading may be made directly (with the naked eye) or through a reading apparatus.

Obviously, various other modifications may be made to the invention within the framework of the appended claims.

1. A system for rapid analysis of a capillary blood sample from a subject, intended for detecting the presence of at least one analyte in said capillary blood sample,
wherein said rapid analysis system comprises:
an analysis module in which is incorporated at least one immunochromatographic strip, designed to detect the presence of said at least one analyte by an immunochromatographic technique,
wherein said at least one immunochromatographic strip includes a deposition area intended to receive said capillary blood sample and a capture area intended to detect the presence of said at least one analyte,
wherein said analysis module includes:
a pricking member, suitable for generating a drop of capillary blood, and
a collecting member, comprising a fixed duct, suitable for capillary blood to flow therethrough,
said duct having an inlet suitable for collecting said drop of capillary blood, and an outlet, arranged opposite said deposition area and suitable for depositing said drop of capillary blood onto said deposition area, and
a container, separate from/independent of said analysis module, in which is packed a buffer solution suitable for implementing said immunochromatographic technique.

2. The system for rapid analysis of a capillary blood sample, according to claim 1, wherein the analysis module has an elongated shape delimited by two ends,
wherein the pricking member is implanted at a first end of said analysis module, and
wherein the collecting member is implanted at a second end of the analysis module.

3. The system for rapid analysis of a capillary blood sample, according to claim 1, wherein the analysis module includes a cassette into which is incorporated said at least one immunochromatographic strip,
and wherein the pricking member and the collecting member are assembled to said cassette through assembly means.

4. The system for rapid analysis of a capillary blood sample, according to claim 3, wherein the pricking member and the collecting member are carried by at least one connecting part.

5. The system for rapid analysis of a capillary blood sample, according to claim 4, wherein the pricking member and the collecting member are carried by:
a single connecting part, which includes means for assembly to the cassette, or
two distinct connecting parts, each including means for assembly to the cassette.

6. The system for rapid analysis of a capillary blood sample, according to claim 4, wherein the pricking member cooperates with said at least one connecting part through assembly means.

7. The system for rapid analysis of a capillary blood sample, according to claim 4, wherein the cassette has two front walls that are connected by a lateral wall, wherein a first front wall includes two windows:
a deposition window, arranged opposite the deposition area of said at least one immunochromatographic strip, and
a reading window, arranged opposite the capture area of said at least one immunochromatographic strip,
and wherein said at least one connecting part has at least:
a front wall placed on said first front wall of the cassette and
a skirt placed on said lateral wall of the cassette.

8. The system for rapid analysis of a capillary blood sample, according to claim 7, wherein the pricking member is implanted opposite and in the continuation of the lateral wall of the cassette.

9. The system for rapid analysis of a capillary blood sample, according to claim 1, wherein the collecting member also includes a through-hole suitable for receiving the buffer solution, arranged opposite the deposition area.

10. The system for rapid analysis of a capillary blood sample, according to claim 9, wherein the through-hole includes an annular surface delimited by an inlet edge and an outlet edge, connected by a flared annular surface.

11. The system for rapid analysis of a capillary blood sample, according to claim 9, wherein the outlet of the duct leads to the through-hole.

12. The system for rapid analysis of a capillary blood sample, according to claim 11, wherein the duct has a gutter shape, whose outlet is cantilevered/protruding with respect to the through-hole.

13. An analysis module for a system for rapid analysis of a capillary blood sample from a subject, intended for detecting the presence of at least one analyte in said capillary blood sample,
wherein said analysis module incorporates at least one immunochromatographic strip designed to detect the presence of said at least one analyte by an immunochromatographic technique,
wherein said at least one immunochromatographic strip includes a deposition area intended to receive said biological sample and a capture area intended to detect the presence of said at least one analyte,
wherein said analysis module includes:
a pricking member, suitable for generating a drop of capillary blood, and
a collecting member, comprising a fixed duct, suitable for capillary blood to flow therethrough, said duct having an inlet for collecting said drop of capillary blood, and an outlet for depositing said drop of capillary blood onto said deposition area,
wherein said analysis module is devoid of container in which is packed a buffer solution suitable for implementing said immunochromatographic technique.

14. The system for rapid analysis of a capillary blood sample, according to claim 9, wherein the collecting member also includes a through-hole suitable for receiving the buffer solution, arranged opposite the deposition area, in said at least one connecting part and opposite the deposition window.

15. The system for rapid analysis of a capillary blood sample, according to claim 3, wherein the pricking member and the collecting member are carried by at least one connecting part, forming an adapter, which cooperate with said cassette through said assembly means.
